(19) United States
(12) Patent Application Publication
(10) Pub. No.: US 2016/0271335 A1
(43) Pub. Date: Sep. 22, 2016

(54) Drive Mechanism for an Injection Device

(71) Applicant: NOVO NORDISC A/S, Bagsvaerd (DK)

(72) Inventors: Lee Higson, Tisvilde (DK); Imran Ghulam, Copenhagen SV (DK); Jesper Bach Noergaard, Virum (DK); Ken Hansen, Virum (DK); Claus Urup Gjoedesen, Copenhagen O (DK); Carsten Schau Andersen, Valby (DK); Asger Meng Larsen, Valby (DK)

(21) Appl. No.: 15/035,651

(22) PCT Filed: Nov. 13, 2014
(86) PCT No.: PCT/EP2014/074483

§ 371 Date: May 10, 2016

Related U.S. Application Data
(60) Provisional application No. 61/907,488, filed on Nov. 22, 2013.

(30) Foreign Application Priority Data
Nov. 20, 2013 (EP) 13193689.0

(51) Int. Cl. A61M 5/315

(52) U.S. Cl. CPC A61M 5/31586; A61M 5/31513

(57) ABSTRACT

The invention relates to a drive mechanism for a medical injection device in which a piston rod (10) has a threaded outer surface and a not circular cross-section and engages a nut assembly (2, 102, 202) having either an internal thread (41, 141, 241) mating the outer thread (11) of the piston rod (10), or a shape mating the non-circular cross section of the piston rod (10). The nut assembly (2) comprises a first part (30, 130, 230) and a second part (40, 140, 240), which are connected by a hinge allowing the first part (30, 130, 230) and the second part (40, 140, 240) to tilt relatively to each other in a non-axial plane.

### THE TECHNICAL FIELD OF THE INVENTION

The invention relates to an injection device for expelling doses of a liquid drug. The invention especially relates to the drive mechanism for such injection device and more especially to such drive mechanism encompassing a nut and thread connection for advancing a piston rod.

### DESCRIPTION OF RELATED ART

An injection device usually has a housing storing a cartridge containing the liquid drug to be injected. The liquid drug is pressed out from the cartridge through an injection needle by moving a plunger forward inside the cartridge. This forward movement is usually done by a piston rod abutting the plunger which piston rod is moved axially forward by a drive mechanism.

An example of such injection devices are disclosed in WO 99/38554 (reference is especially made to the FIGS. 15-17, which is currently sold by Novo Nordisk A/S under the trade name Flexpen®) and in US 2011/0054412. The dosing mechanism disclosed comprises a piston rod having an external thread which is engaged in an internal thread which is provided in a nut member firmly secured in the housing. A drive tube engages a longitudinal track provided in the piston rod such that when the drive tube is rotated, the piston rod rotates with it and is screwed axially forward in the internal thread of the nut member.

In WO 99/38554, the drive tube is forced to rotate when an injection button is pressed home into the housing.

The injection device disclosed in US 2011/0054412 is a so-called automatic injection device i.e. an injection device utilizing a spring to expel the liquid drug. In this injection device, the drive tube is forced to rotate by a torsion spring which is strained during dose setting. The rotation of the drive tube screws the piston rod forward through the nut member which is secured to the housing.

A different automatic injection device is disclosed in WO 2011/003979 (especially in FIGS. 20A-B). In this injection device which is sold by Novo Nordisk A/S under the trade name FlexTouch®, the piston rod is also surrounded by a tube which is used to reset a selected dose. The piston rod is threaded to a nut member secured in the housing and the non-circular cross section of the piston rod is engaged by a transfer clutch. The piston rod is thus screwed forward when the transfer clutch is rotated relatively to the nut member.

When manufacturing injection devices various tolerances apply, both on the moulding of the individual parts and in the assembly of the individual parts. Due to these tolerances there is a risk that the nut member is positioned in an off-centre position such that the piston rod is not moved forward along the centre line of the injection device but is deflected from the centre line. The nut member could e.g. be mounted such that the thread it is not 100 percent perpendicular to the centre line. As a result, the proximal end of the piston rod could slide against the inside wall of a part surrounding the piston rod and thus create parasitic loads as schematically depicted in FIG. 1. Parasitic loads could also arise in the thread.

Further, the moulding of the thread either on the piston rod or inside the nut member (or both) could be imprecise which again could lead to a deflection of the piston rod.

Sometime the nut member is moulded integrally with the housing, however also in this case the tolerances could be such that the piston rod deflects during its axial movement.

In order to minimize the outside diameter of the injection device the different layers making up the mechanism of the injection device needs to be positioned very close to each other which minimize the possible deflection of the piston rod without generating parasitic loads.

Throughout this specification the general term “parasitic loads” is used to describe any kind of unwanted loss of forces. The major loss occurs due to unintended friction as the piston rod slides against an internal surface of a relatively stationary component i.e. a component stationary relatively to the movement of the piston rod.

If the injection device is a spring loaded automatic injection device as in US 2011/0054412 the force of the spring must be sufficient to overcome these unwanted parasitic loads. In a manual injection device the force to be delivered by the user needs to be higher in order to overcome these loads.

In order to minimize production cost it is desirable to allow large tolerances, however this requires that these unwanted parasitic loads can be removed or at least reduced.

### DESCRIPTION OF THE INVENTION

It is thus an object of the present invention to provide a drive mechanism for an injection device which removes or at least dramatically reduces parasitic loads thereby allowing the use of moulded parts having relatively large tolerances.

Such drive mechanism must preferably be workable both in manual and in automatic injection devices.

The invention is defined in claim 1. Accordingly in one aspect the present invention relates to a drive mechanism for a medical injection device.

Such drive mechanism usually comprises a piston rod and a nut assembly. The piston rod has a threaded outer surface and a not circular cross-section, and the nut assembly has either an internal thread mating the outer thread of the piston rod, or an internal shape mating the non-circular cross section of the piston rod.

Usually the piston rod is also engaged by a drive element which either has a thread mating the thread of the piston rod or an internal shape mating the non-circular cross section of the piston rod.

The liquid drug of the injection device is usually contained in a cartridge which at one end has a plunger that can be moved forward by the axial movement of the piston rod and at the opposite end has an injection needle through which the liquid drug can flow as the plunger is moved forward.

Further, in an injection device a mechanism is provided which is able to rotate the drive element an angle relating to the size of the dose to be expelled. The rotation of the drive element thus forces the piston rod to move in the distal direction.

If the piston rod is threaded to the nut assembly it is usually keyed to the drive element such that rotation of the drive element causes the piston rod to be rotated and thus screwed forward in a rotational and spiral movement.

If the piston rod is keyed to the nut assembly it is usually threaded to the drive element such that rotation of the drive element causes the piston rod to move axially forward without rotation

In both cases, relative rotation between the piston rod and the nut assembly causes the piston rod to move axially.

According to a first embodiment, the nut assembly comprises a first part and a second part, which are connected by a hinged connection allowing the first part and the second part to tilt in relatively to each other, thus allowing the piston rod to deflect from the centre line of the injection device without creating parasitic loads.

By tilt is meant that the two parts are pivotally mounted and thus able to move in relation to each other in one plane around a fixed axis, similar to the tilting movement of a seesaw, however, the fixed axis does not need to follow the horizontal plane, but can be any plane determined by the position of three points in space.

This hinged connection makes it possible for the centre axis (Y) of the second part and the centre axis of the first part (X) to deflect relatively to each other without creating parasitic loads.

The first part is both rotational and axially locked to the housing such that the centre axis (X) of the first part follows the centre axis of the injection device. Since the second part is allowed to tilt, the second centre axis (Y) of the second part is allowed to deflect from the first centre axis (X).

In one example, the first part could be integral with the housing. The first part could e.g. moulded in one with the housing or it could be a separate part connected to the housing of the injection device such that the first part and the housing operate as one and the same component.

In one example, the second part is rotational locked to the first part such that the piston rod is moved forward whenever a drive element engaging the piston rod is rotated. When the second part is rotational locked to the first part which again is both rotational and axilly locked to the housing, the second part is also rotational locked to the housing thus the first part and the second part together form one rotational stationary nut assembly.

The second part is preferably also axially secured to the first part. This connection in the axial direction can in one preferred embodiment be formed as a ball and socket connection allowing the second part to tilt in relation to the first part thus preventing relative axial movement of the two parts.

The hinged connection is preferably made by the second part having a number of protrusions engaging similar cut-outs in the first part. Preferably, two such protrusions are provided 180 degrees apart such that the protrusions lies on the same axis perpendicular to the centre axis of the injection device thus being the axis around which the second part can tilt. The protrusion and the cut-outs also hinder the second part from rotating relatively to the first part.

In a second embodiment a third and a fourth part is provided between the first part and the second part thereby making it possible for the second part to simultaneously tilt and pan in relation to the first part.

By pan is also meant to perform a pivotal movement in one plane around a fixed axis. However, the term “tilt and pan” indicates that the axis is displaced in relation to the axis around which it tilts. The displacement is preferably approximately 90 degrees such that the two axes are approximately perpendicular to each other, however any angular displacement between the two planes are intended to be covered by the claims.

In the second embodiment, the second part is non-movable fitted to the third part e.g. by being moulded as one element or by being click- or press fitted together.

The third part is then hinged to the fourth part which again is hinged to the first part. The fourth part can thus tilt in relation to the first part and the third part can pan in relation to the fourth part.

The principle of incorporating a mechanism in an injection device which allows components to tilt and pan relatively to each other in e.g. two radial directions maintaining the possibility of transfer a torque can be usable in other position in the injection device where parasitic loads should be avoided. E.g. the clutch mechanism transferring the torque in a torsion spring device could be made as a cardan joint or an Oldham joint.

The invention further includes an injection device utilizing such a drive mechanism.

As explained above such injection device usually has a drive element which engages the piston rod either by thread or by a non-circular cross section. Whenever such drive element is rotated the piston rod performs an axial movement. This axial movement can either be a purely axial movement or it can be combined with a rotational movement resulting in a helical movement.

### Definitions

An “injection pen” is typically an injection apparatus having an oblong or elongated shape somewhat like a fountain pen for writing. Although such pens usually have a tubular cross-section, they could easily have a different cross-section such as triangular, rectangular or square or any variation around these geometries.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container containing the drug. Cartridges are usually made from glass but could also be moulded from any suitable polymer. A cartridge or ampoule is preferably sealed at one end by a pierceable membrane referred to as the “septum” which can be pierced e.g. by the back-end of a needle cannula. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Further the term “injection needle” defines a piercing member adapted to penetrate the skin of a subject for the purpose of delivering or removing a liquid. For many pen systems, the needle cannula of the injection needle comprises a front part for penetrating the skin of the user and a back part for penetrating the septum of the cartridge thus creating a liquid flow between the interior of the cartridge and the subcutaneous layer of the user.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show an example of the prior art.

FIG. 2 show the principle of the present invention.

FIG. 3 show a cross-sectional view of a first embodiment.

FIG. 4 show a perspective view with a part cut open of the first embodiment.

FIG. 5 show an exploded view of the embodiment in FIG. 3.

FIG. 6 show a cross-sectional view of a second embodiment.

FIG. 7 show a perspective view with a part cut open of the second embodiment.

FIG. 8 show an exploded view of the embodiment in FIG. 6.

FIG. 9 show an end view of a third embodiment of the invention.

FIGS. 10A-B show views of yet another embodiment of the invention.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

In that context it may be convenient to define that the term “distal end” in the appended figures is meant to refer to the end of the injection device which usually carries the injection needle whereas the term “proximal end” is meant to refer to the opposite end pointing away from the injection needle and usually carrying the dose dial button.

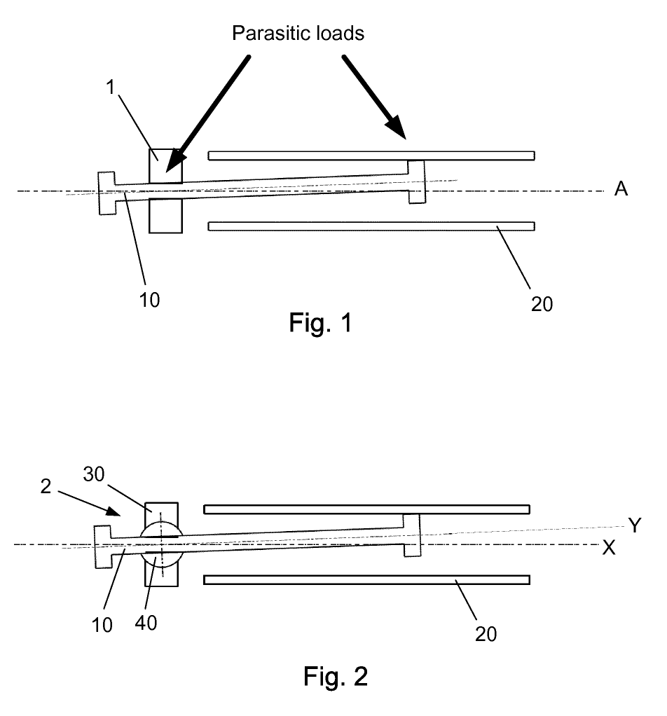

FIG. 1 discloses a schematic view of the prior art. The nut member 1 is usually non rotatable connected to the housing of the injection device such that it cannot move in any direction. Alternatively, the nut member 1 is moulded integral with the housing of the injection device.

The nut member 1 is internally provided with a thread. A piston rod 10 which externally is provided with a thread is threaded to the internal thread of the nut member 1 such that the piston rod 10 is screwed forward when rotated. The piston rod 10 thus performs a helical movement.

A tube 20 surrounds the piston rod 10. This tube 20 could be a drive tube, a reset tube or just simply a layer of the mechanism. The tube 20 is typically stationary in relation to the piston rod 10 at least in an axial direction i.e. as the piston rod 10 is moved forward.

If the piston rod 10 deflects from the centre line A of the injection device e.g. due to tolerances, the proximal end of the piston rod 10 could abut or slide on the internal surface of the tube 20 and thus create parasitic loads when moving forward. Further parasitic loads can be generated in the thread connection due to this deflection.

FIG. 2 discloses a solution according to the present invention wherein the nut assembly 2 now comprises of a first part 30 which is firmly fitted to the housing and a second part 40 which carries the thread for screwing forward the piston rod 10. The second part 40 is connected to the first part 30 via a hinge such that the first part 30 can tilt relatively to the second part 40 at least in one plane.

The first part 30 has a first centre axis X and the second part 40 has a second centre line Y. As indicated in FIG. 2, the hinged connection between the first part 30 and the second part 40 allows the first centre axis X and the second centre axis Y to deflect from each other. This hinge operating between the first part 30 and the second member 40 thus allows deflection of the piston rod 10 without creating parasitic loads as will be explained in details going forward.

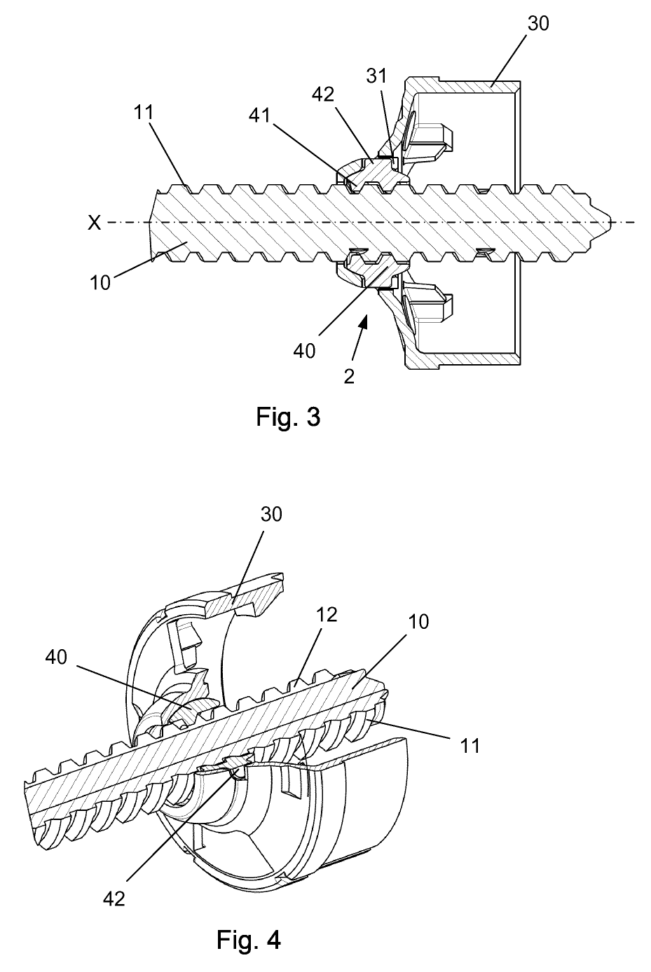

A cross-sectional view is disclosed in FIG. 3. The first part 30 is firmly connected to a not shown housing such that the first part 30 is stationary relatively to the housing. The first centre axis X of the first part 30 thus usually follows the centre axis A of the injection device.

The piston rod 10 is externally provided with a longitudinal thread 11 which engages an internal thread 41 provided in the second part 40. The piston rod 10 further has a non-circular cross-section 12 to be gripped by a not-showed drive element.

Further as disclosed in FIG. 4, the second part 40 is provided with a number of protrusions 42 making up the hinge. These protrusions 42 rest in similar cut-outs 31 in the first part 30. Preferably, there are two protrusions 42 and two cut-outs 31 which are situated 180 degrees dislocated such that the second part 40 can tilt in one plane in relation to the first part 30. Further, in this way the second part 40 is rotational locked to the first part 30 such that the piston rod 10 rotates forward in the thread 41 when the piston rod 10 is rotated relatively to the nut assembly 2.

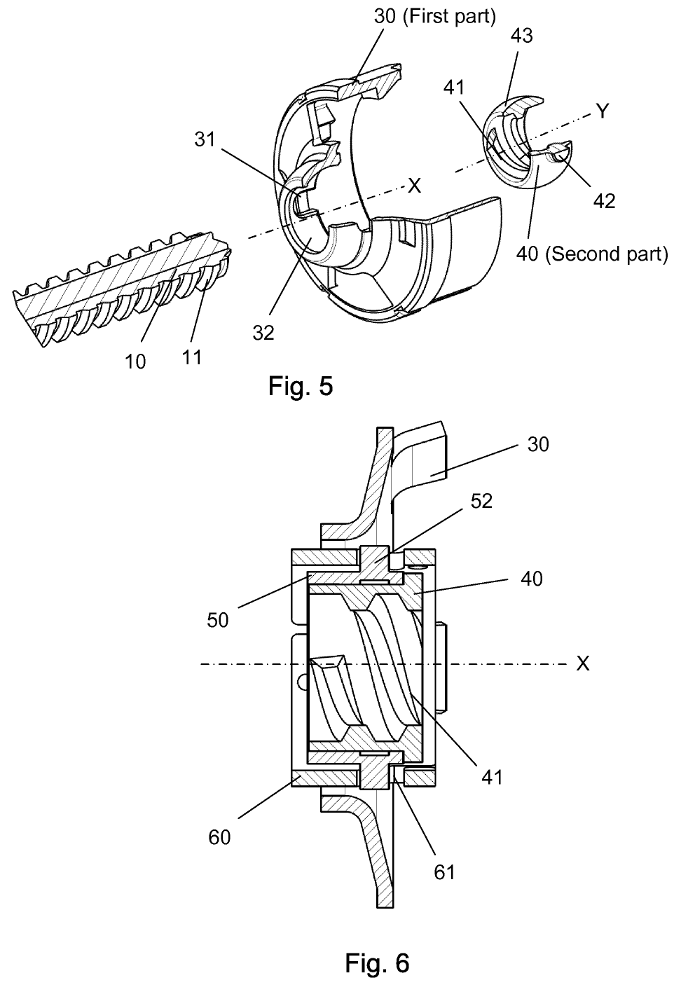

The first part 30 having a first centre axis X and the second part 40 having a second centre axis Y is disclosed in details in FIG. 5.

As further depicted in FIG. 5, the outer wall 43 of the second part 40 is preferably ball-shaped and press fitted into a similar cup-shaped surface 32 in the first part 30 making up a ball-and-socket connection such that the first part 30 and the second part 40 stay axially connected, i.e. the first part 30 cannot move axially relatively to the second part 40. Thus, the second part 40 can only rotate relatively to the first part 30 in this coupling; however, this rotational movement is prevented due to the protrusions 42 engaging the cut-outs 31. The only resulting allowable movement between the second part 40 and the first part 30 is thus a tilting of the second part 40 in one plane relatively to the first part 30. The plane is defined by a common axis through the two protrusions 42

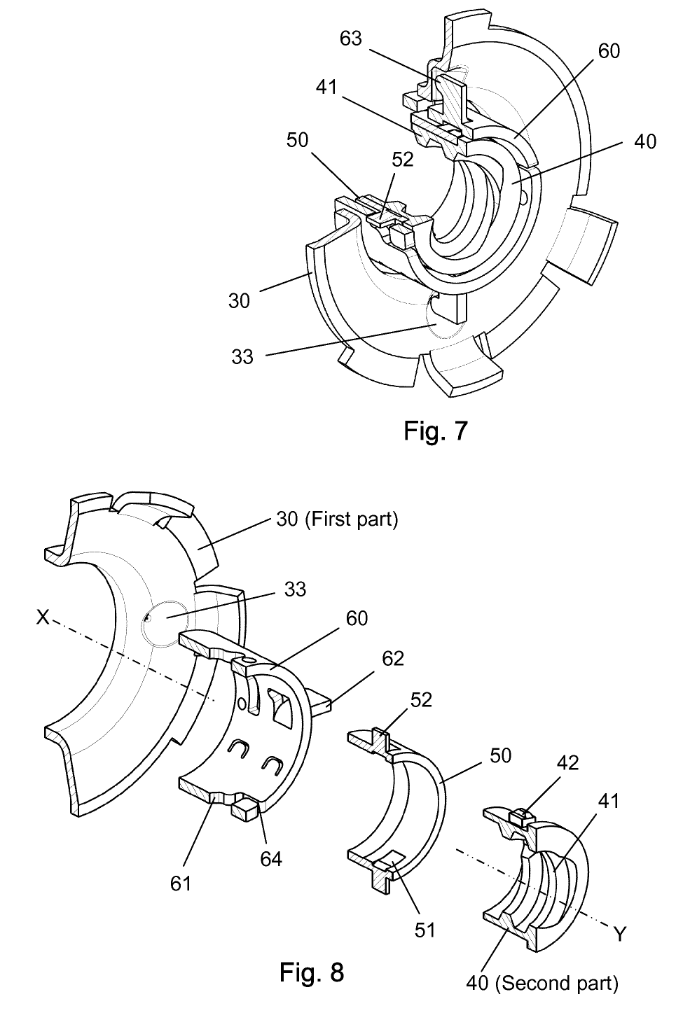

The FIGS. 6 to 8 disclose a different embodiment in which the same reference numbers are used for similar parts.

The piston rod (which is not shown in this second embodiment) has an external thread which engages a thread 41 in the second part 40. Further, the first part 30 is firmly secured in the not shown housing. As in the first embodiment, the first part 30 has a first centre axis X and the second part has a second centre axis 40.

The second part 40 carrying the thread 41 is secured to a third part 50 by having a number of protrusions 42 engaging similar indentations 51 in the third part 50. The second part 40 and the third part 50 is in this way connected to each other both axially and rotational such that the second part 40 cannot move in any direction relatively to the third part 50. Alternatively the second part 40 and the third part 50 can be produced as one single element.

Further, the third part 50 is provided with external protrusions 52 which engage similar notches 61 in the fourth part 60. There are preferably two such protrusions 52 located 180 degrees apart such that the third part 50 can tilt or pan relatively to the fourth part 60.

The fourth part 60 is in the same manner provided with external arms 62 having a distally pointing extension 63 which rest in a cup- shaped deformation 33 formed in the first part 30. The extension 63 is preferably pushed into the cup 33 by a not shown pressure element or the like. These arms 62 are also preferably provided as a pair which is 180 degrees dislocated such that the fourth part 60 can tilt or pan relatively to the first part 30.

The notches 61 preferably have an S-shaped entrance 64 such that the third part 50 cannot move axially in relation to the fourth part 60 when properly mounted.

All together the third part 50 and the fourth part 60 makes it possible for the second part 40 to move in two planes relatively to the first part 30. The second part 40 can thus simultaneous both tilt and pan relatively to the first part 30. At the same time, the second part 40 is rotational and axially locked to the first part 30 and thus to the housing.

In this way, the piston rod 10 can simultaneously tilt and pan relatively to centre line X. The first 30, second 40, third 50 and fourth part 60 can be made from any suitable material e.g. a polymeric material; however the first part 30, the third part 50 and the fourth part 60 are preferably made from a metallic material.

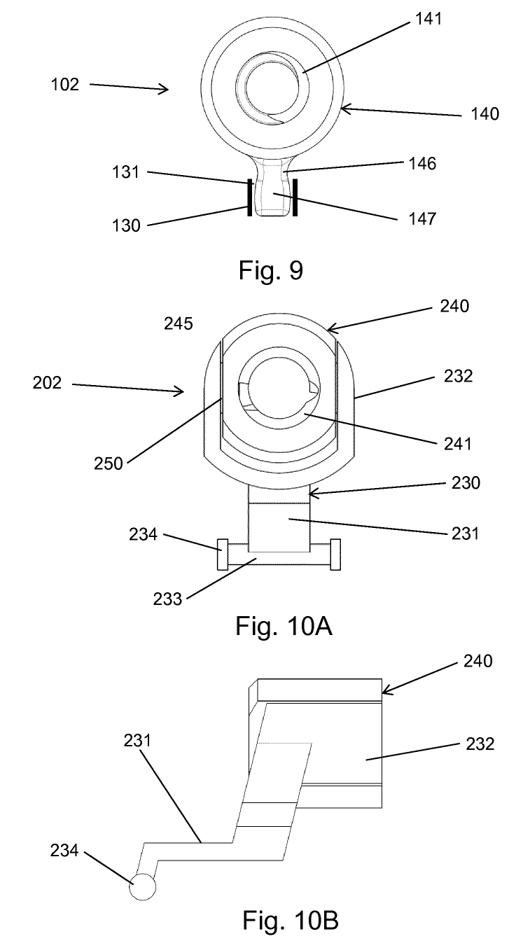

FIG. 9 shows a further embodiment of a nut assembly, and the FIGS. 10A and 10B show a yet further embodiment of a nut assembly.

Whereas the embodiments of FIGS. 3-8 have a generally cylindrical design making them suitable for incorporation in pen-shaped drug delivery devices having a general cylindrical configuration (as well as in devices having a non-cylindrical configuration), the embodiments of FIGS. 9 and 10 have not been optimized for incorporation in a pen-shaped drug delivery device in which a small diameter is desirable. Correspondingly, the embodiments of FIGS. 9 and 10 may be more suitable for drug delivery devices having a box-shaped configuration, e.g. in the form of a “dozer” which may be manually actuated or motorized.

Referring to FIG. 9, a nut assembly 102 is shown comprising a first part and a second part. The first part which is only shown schematically is in the form of a base member 130 comprising an opening or bore 131. The base member may be formed integrally with the general housing of the drug delivery device or a separate member attached to the housing. The second part is in the form of a nut member 140 comprising a cylindrical main portion 145 with a central bore defining a Z-axis (arranged perpendicular to the plane of the figure), the central bore being provided with a thread 141 adapted to receive and engage a correspondingly threaded piston rod. The second part further comprises a rod-like portion 146 extending radially from the exterior surface of the main portion. The rod comprises a free distal end with an enlarged head portion 147 adapted to be received in sliding engagement with the base member bore 131, this providing a rotational lock of the nut member relative to the base member in respect of the Z-axis yet allows the nut member to move axially in respect of the Y-axis as shown. Further, as the head portion is received in the bore with a small amount of slack the nut member is allowed to move a small amount also in respect of the X-axis just it is allowed to pivot a small amount in the X-Y plane.

Referring to FIGS. 10A and 10B a nut assembly 202 is shown comprising a first part and a second part. The first part is in the form of a fork member 230 comprising a body portion 231, a pair of opposed wall portions 232, and a hinge pin portion 233. The second part is in the form of a nut member 240 comprising a generally cylindrical main portion 245 with a central bore defining a Z-axis (arranged perpendicular to the plane of FIG. 10A), the central bore being provided with a thread 241 adapted to receive and engage a correspondingly threaded piston rod. On the exterior the nut member comprises a pair of opposed co-planer flat portions adapted to be received between the wall portions of the fork member, a combined first hinge 250 being formed there between allowing the nut member to pivot corresponding to a Y-axis arranged in the plane of FIG. 10A. In the shown embodiment a pivotal hinge is formed on each side by an axle extending inwardly from the wall which is received in a corresponding nut wall cavity. The pin portion 233 is adapted to be received in a corresponding hinge structure, e.g. formed by a housing member (not shown). In the shown embodiment the formed second hinge allows the fork member to both pivot in respect of the X-axis just as it is allowed to move axially a small amount in the X-Y plane. In the shown embodiment the pin portion 233 is provided with enlarged end portions 234 providing stops for axial movement. The combined freedom of the two hinges allow the nut member to move in the Y-direction, albeit not axially. Further, the two hinges provide a rotational lock of the nut member relative to the fork member respectively to a housing member.

Some preferred embodiments have been shown in the foregoing, but it should be stressed that the invention is not limited to these, but may be embodied in other ways within the subject matter defined in the following claims.

1. A drive mechanism for a medical injection device comprising:
a piston rod having a threaded outer surface and a not circular cross-section,
a nut assembly having an internal thread mating the outer thread of the piston rod, or an internal shape mating the non-circular cross section of the piston rod, such that relative rotation between the nut assembly and the piston rod drives the piston rod axially, wherein,
the nut assembly comprises a first part and a second part, which first part and second part are connected by a hinged connection allowing the first part and the second part to tilt in relation to each other.

2. A drive mechanism according to claim 1, wherein the first part has a first centre axis (X) and the second part has a second centre axis (Y) and wherein the first part and the second part is tiltable in relation to each other from a first position in which the second centre axis (Y) is parallel to the first centre axis (X), to a second position in which the first axis (Y) and the second axis (X) is non-parallel.

3. A drive mechanism according to claim 1, wherein the first part is rotational and axially locked to the housing.

4. A drive mechanism according to claim 3, wherein the second part is rotational locked in relation to the first part.

5. A drive mechanism according to claim 1, wherein the second part is axially secured in relation to the first part.

6. A drive mechanism according to claim 1, wherein the second part is threaded to the piston rod or engages the non-circular cross-section of the piston rod.

7. A drive mechanism according to claim 1, wherein the second part has a number of protrusion engaging similar cut-outs in the first part.

8. A drive mechanism according to claim 1, wherein the second part is fitted in a third part.

9. A drive mechanism according to claim 8, wherein the third part is hinged to a fourth part.

10. A drive mechanism according to claim 9, wherein the fourth part is hinged to the first part.

11. A drive mechanism according to claim 9, wherein the third part is provided with a number of protrusions engaging similar indentations in the fourth part.

12. A drive mechanism according to claim 9, wherein the fourth part has a number of arms engaging cups provided in the first part.

13. An injection device comprising a drive mechanism as in claim 1.

14. An injection device according to claim 13, wherein a drive element engages the piston rod and wherein the piston rod is driven in the distal direction upon rotation of the drive element.
